SEQUENCE LISTING (16 sequences)

SEQ ID NO: 1 - DNA, 807 bp, Thermomyces lanuginosus
  CDS (1)..(807)
  mat_peptide (1)..()
gaggtctcgcaggatctgtttaaccagttcaatctctttgcacagtattctgcagccgcatactgcggaaaaaacaatgatgccccagctggtacaaacattacgtgcacgggaaatgcctgccccgaggtagagaaggcggatgcaacgtttctctactcgtttgaagactctggagtgggcgatgtcaccggcttccttgctctcgacaacacgaacaaattgatcgtcctctctttccgtggctctcgttccatagagaactggatcgggaatcttaacttcgacttgaaagaaataaatgacatttgctccggctgcaggggacatgacggcttcacttcgtcctggaggtctgtagccgatacgttaaggcagaaggtggaggatgctgtgagggagcatcccgactatcgcgtggtgtttaccggacatagcttgggtggtgcattggcaactgttgccggagcagacctgcgtggaaatgggtatgatatcgacgtgttttcatatggcgccccccgagtcggaaacagggcttttgcagaattcctgaccgtacagaccggcggaacactctaccgcattacccacaccaatgatattgtccctagactcccgccgcgcgaattcggttacagccattctagcccagagtactggatcaaatctggaacccttgtccccgtcacccgaaacgatatcgtgaagatagaaggcatcgatgccaccggcggcaataaccagcctaacattccggatatccctgcgcacctatggtacttcgggttaattgggacatgtctt

SEQ ID NO: 2 - protein, 269 aa, Thermomyces lanuginosus [sequence omitted]

SEQ ID NO: 3 - protein, 265 aa, Absidia reflexa
SSSSTQDYRIASEAEIKAHTFYTALSANAYCRTVIPGGRWSCPHCGVASNLQITKTFSTLITDTNVLVAVGEKEKTIYVVFRGTSSIRNAIADIVFVPVNYPPVNGAKVHKGFLDSYNEVQDKLVAEVKAQLDRHPGYKIVVTGHSLGGATAVLSALDLYHHGHANIEIYTQGQPRIGTPAFANYVIGTKIPYQRLVHERDIVPHLPPGAFGFLHAGEEFWIMKDSSLRVCPNGIETDNCSNSIVPFTSVIDHLSYLDMNTGLCL

SEQ ID NO: 4 - protein, 264 aa, Absidia corymbifera
SSSTQDYRIASEAEIKAHTFYTALSANAYCRTVIPGGQWSCPHCDVAPNLNITKTFTTLITDTNVLVAVGENEKTIYVVFRGTSSIRNAIADIVFVPVNYPPVNGAKVHKGFLDSYNEVQDKLVAEVKAQLDRHPGYKIVVTGHSLGGATAVLSALDLYHHGHDNIEIYTQGQPRIGTPEFANYVIGTKIPYQRLVNERDIVPHLPPGAFGFLHAGEEFWIMKDSSLRVCPNGIETDNCSNSIVPFTSVIDHLSYLDMNTGLCL

SEQ ID NO: 5 - protein, 269 aa, Rhizomucor miehei
SIDGGIRAATSQEINELTYYTTLSANSYCRTVIPGATWDCIHCDATEDLKIIKTWSTLIYDTNAMVARGDSEKTIYIVFRGSSSIRNWIADLTFVPVSYPPVSGTKVHKGFLDSYGEVQNELVATVLDQFKQYPSYKVAVTGHSLGGATALLCALDLYQREEGLSSSNLFLYTQGQPRVGDPAFANYVVSTGIPYRRTVNERDIVPHLPPAAFGFLHAGEEYWITDNSPETVQVCTSDLETSDCSNSIVPFTSVLDHLSYFGINTGLCT

SEQ ID NO: 6 - protein, 271 aa, Rhizopus oryzae
SASDGGKVVAATTAQIQEFTKYAGIAATAYCRSVVPGNKWDCVQCQKWVPDGKIITTFTSLLSDTNGYVLRSDKQKTIYLVFRGTNSFRSAITDIVFNFSDYKPVKGAKVHAGFLSSYEQVVNDYFPVVQEQLTAHPTYKVIVTGHSLGGAQALLAGMDLYQREPRLSPKNLSIFTVGGPRVGNPTFAYYVESTGIPFQRTVHKRDIVPHVPPQSFGFLHPGVESWIKSGTSNVQICTSEIETKDCSNSIVPFTSILDHLSYFDINEGSCL

SEQ ID NO: 7 - protein, 267 aa, Aspergillus niger
TAGHALAASTQGISEDLYSRLVEMATISQAAYADLCNIPSTIIKGEKIYNSQTDINGWILRDDSSKEIITVFRGTGSDTNLQLDTNYTLTPFDTLPQCNGCEVHGGYYIGWVSVQDQVESLVKQQVSQYPDYALTVTGHSLGASLAALTAAQLSATYDNIRLYTFGEPRSGNQAFASYMNDAFQASSPDTTQYFRVTHANDGIPNLPPVEQGYAHGGVEYWSVDPYSAQNTFVCTGDEVQCCEAQGGQGVNNAHTTYFGMTSGACTW

SEQ ID NO: 8 - protein, 266 aa, Aspergillus tubingensis
TAGHALAASTQGISEDLYSRLVEMATISQAAYADLCNIPSTIIKGEKIYNSQTDINGWILRDDSSKEIITVFRGTGSDTNLQLDTNYTLTPFDTLPQCNSCEVHGGYYIGWISVQDQVESLVQQQVSQFPDYALTVTGHSLGASLAALTAAQLSATYDNIRLYTFGEPRSNQAFASYMNDAFQASSPDTTQYFRVTHANDGIPNLPPADEGYAHGVVEYWSVDPYSAQNTFVCTGDEVQCCEAQGGQGVNNAHTTYFGMTSGHCTW

SEQ ID NO: 9 - protein, 276 aa, Fusarium oxysporum
AVGVTTTDFSNFKFYIQHGAAAYCNSEAAAGSKITCSNNGCPTVQGNGATIVTSFVGSKTGIGGYVATDSARKEIVVSFRGSINIRNWLTNLDFGQEDCSLVSGCGVHSGFQRAWNEISSQATAAVASARKANPSFNVISTGHSLGGAVAVLAAANLRVGGTPVDIYTYGSPRVGNAQLSAFVSNQAGGEYRVTHADDPVPRLPPLIFGYRHTTPEFWLSGGGGDKVDYTISDVKVCEGAANLGCNGGTLGLDIAAHLHYFQATDACNAGGFSWRR

SEQ ID NO: 10 - protein, 273 aa, Fusarium heterosporum
TVTTQDLSNFRFYLQHADAAYCNFNTAVGKPVHCSAGNCPDIEKDAAIVVGSVVGTKTGIGAYVATDNARKEIVVSVRGSINVRNWITNFNFGQKTCDLVAGCGVHTGFLDAWEEVAANVKAAVSAAKTANPTFKFVVTGHSLGGAVATIAAAYLRKDGFPFDLYTYGSPRVGNDFFANFVTQQTGAEYRVTHGDDPVPRLPPIVFGYRHTSPEYWLNGGPLDKDYTVTEIKVCEGIANVMCNGGTIGLDILAHITYFQSMATCAPIAIPWKR

SEQ ID NO: 11 - protein, 278 aa, Aspergillus oryzae
DIPTTQLEDFKFWVQYAAATYCPNNYVAKDGEKLNCSVGNCPDVEAAGSTVKLSFSDDTITDTAGFVAVDNTNKAIVVAFRGSYSIRNWVTDATFPQTDPGLCDGCKAELGFWTAWKVVRDRIIKTLDELKPEHSDYKIVVVGHSLGAAIASLAAADLRTKNYDAILYAYAAPRVANKPLAEFITNQGNNYRFTHNDDPVPKLPLLTMGYVHISPEYYITAPDNTTVTDNQVTVLDGYVNFKGNTGTSGGLPDLLAFHSHVWYFIHADACKGPGLPLR

SEQ ID NO: 12 - protein, 278 aa, Penicillium camemberti
DVSTSELDQFEFWVQYAAASYYEADYTAQVGDKLSCSKGNCPEVEATGATVSYDFSDSTITDTAGYIAVDHTNSAVVLAFRGSYSVRNWVADATFVHTNPGLCDGCLAELGFWSSWKLVRDDIIKELKEVVAQNPNYELVVVGHSLGAAVATLAATDLRGKGYPSAKLYAYASPRVGNAALAKYITAQGNNFRFTHTNDPVPKLPLLSMGYVHVSPEYWITSPNNATVSTSDIKVIDGDVSFDGNTGTGLPLLTDFEAHIWYFVQVDAGKGPGLPFKR

SEQ ID NO: 13 - protein, 270 aa, Aspergillus foetidus
SVSTSTLDELQLFAQWSAAAYCSNNIDSKDSNLTCTANACPSVEEASTTMLLEFDLTNDFGGTAGFLAADNTNKRLVVAFRGSSTIENWIANLDFILEDNDDLCTGCKVHTGFWKAWESAADELTSKIKSAMSTYSGYTLYFTGHSLGGALATLGATVLRNDGYSVELYTYGCPRIGNYALAEHITSQGSGANFRVTHLNDIVPRVPPMDFGFSQPSPEYWITSGNGASVTASDIEVIEGINSTAGNAGEATVSVLAHLWYFFAISECLL

SEQ ID NO: 14 - protein, 270 aa, Aspergillus niger
SVSTSTLDELQLFSQWSAAAYCSNNIDSDDSNVTCTADACPSVEEASTKMLLEFDLTNNFGGTAGFLAADNTNKRLVVAFRGSSTIKNWIADLDFILQDNDDLCTGCKVHTGFWKAWEAAADNLTSKIKSAMSTYSGYTLYFTGHSLGGALATLGATVLRNDGYSVELYTYGCPRVGNYALAEHITSQGSGANFPVTHLNDIVPRVPPMDFGFSQPSPEYWITSGTGASVTASDIELIEGINSTAGNAGEATVDVLAHLWYFFAISECLL

SEQ ID NO: 15 - protein, 269 aa, Aspergillus oryzae
DVSSSLLNNLDLFAQYSAAAYCDENLNSTGTKLTCSVGNCPLVEAASTQSLDEFNESSSYGNPAGYLAADETNKLLVLSFRGSADLANWVANLNFGLEDASDLCSGCEVHSGFWKAWSEIADTITSKVESALSDHSDYSLVLTGHSYGAALAALAATALRNSGHSVELYNYGQPRLGNEALATYITDQNKGGNYRVTHTNDIVPKLPPTLLGYHHFSPEYYISSADEATVTTTDVTEVTGIDATGGNDGTDGTSIDAHRWYFIYISECS

SEQ ID NO: 16 - protein, 251 aa, Landerina penisapora
PQDAYTASHADLVKYATYAGLAYQTTDAWPASRTVPKDTTLISSFDHTLKGSSGYIAFNEPCKEIIVAYRGTDSLIDWLTNLNFDKTAWPANISNSLVHEGFLNAYLVSMQQVQEAVDSLLAKCPDATISFTGHSLGGALACISMVDTAQRHRGIKMQMFTYGQPRTGNQAFAEYVENLGHPVFRVVYRHDIVPRMPPMDLGFQHHGQEVWYEGDENIKFCKGEGENLTCELGVPFSELNAKDHSEYPGMH

(19) United States
(12) Patent Application Publication
(10) Pub. No.: US 2007/0179074 A1
(43) Pub. Date: Aug. 2, 2007

(54) Detergent compositions

(76) Inventors: Philip Frank Souter, Northumberland (GB); John Allen Burdis, upon Tyne (GB); Allan Svendsen, Horsholm (DK); Thomas Honger Callisen, Frederiksberg (DK); Jesper Vind, Vaerlose (DK); Debbie Yaver, Davis, CA (US); Jurgen Carsten Franz Knotzel, Coperhagen (DK); Kim Borch, Davis, CA (US); Mads Eskelund Bjornvad, Virum (DK); Peter Kamp Hansen, Lejre (DK); Michael Lamsa, Davis, CA (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(21) Appl. No.: 11/656,254

(22) Filed: Jan. 22, 2007

Related U.S. Application Data
(60) Provisional application No. 60/761,107, filed on Jan. 23, 2006.
(60) Provisional application No. 60/796,268, filed on Apr. 28, 2006.
(60) Provisional application No. 60/854,753, filed on Oct. 27, 2006.

(51) Int. Cl. C11D 3/386; C12N 9/18

(52) U.S. Cl. 510/320; 435/197

(57) ABSTRACT

The present invention relates to detergent compositions comprising a detergent ingredient and a lipase variant with reduced potential for odor generation obtained by introducing mutations in one or more regions identified in a parent lipase.

### CROSS-REFERENCES TO RELATED APPLICATIONS

This application claims priority under 35 U.S.C. § 119(e) to U.S. Provisional Application Ser. No. 60/761,107 filed Jan. 23, 2006, U.S. Provisional Application Ser. No. 60/796,268 filed Apr. 28, 2006, and U.S. Provisional Application Ser. No. 60/854,753 filed Oct. 27, 2006.

### FIELD OF THE INVENTION

The present invention relates to detergent compositions, particularly laundry detergents, comprising lipolytic enzymes.

### BACKGROUND OF THE INVENTION

Improved removal of greasy soils is a constant aim for detergent manufacturers, especially in the laundry context. In spite of the use of many effective surfactants and combinations of surfactants, especially when used at low water temperatures, many surfactant-based products still do not achieve complete removal of greasy/oily soils. Lipase enzymes have been used in detergents since the late 1980s for removal of fatty soils by breakdown of fatty soils into tri-glycerides.

Until relatively recently, the main commercially available lipase enzymes, such as Lipolase (trade name, Novozymes) worked particularly effectively at the lower moisture levels of the drying phase of the wash process. These enzymes tended to produce significant cleaning only in the second wash step with fat breakdown significant only on soils remaining on laundered clothes during the drying stage, the broken down fats then being removed in the next washing step. However, more recently, higher efficiency lipases have been developed that also work effectively during the wash phase of the cleaning process, so that as well as cleaning in the second washing step, a significant improvement in cleaning effect due to lipase enzyme can be found in the first wash-cycle. Examples of such enzymes are as described in U.S. Pat. No. 6,939,702 B1, WO 00/60063 and Research Disclosure IP6553D. Such enzymes are referred to below as first wash lipases.

In addition, consumers prefer that articles, such as garments, be as clean as possible. Such consumers typically associate the odor of a cleaned or treated article with the degree of cleanliness of such article. Thus, the effectiveness of a cleaning and/or treatment composition, from a consumer's perspective, is typically directly linked with the odor that such composition imparts to an article that is cleaned or treated with such composition. Applicants recognized that certain materials, such as esterases and lipases, can generate objectionable fatty acid odors, particularly short-chain fatty acid odors such as the odor of butyric acid. However, such materials can be particularly effective cleaning agents. Unfortunately, consumers typically associate the odors resulting from the use of such agents with a lack of cleanliness. Examples of reduced odour variants with a C-terminal extension are shared in WO02/062973, but these lipase variants do not demonstrate the strong wash performance of the first wash lipases such as those from WO00/60063 including the variant sold under the tradename Lipex®.

Thus, there remains a need for a detergent compositions comprising lipolytic enzymes for excellent greasy/oily soils removal while not generating any objectionable fatty acid odors.

### SUMMARY OF THE INVENTION

The present invention relates to detergent compositions comprising a detergent ingredient and a lipase variant with reduced potential for odor generation, without the attachment of a C-terminal extension. The lipase variant is obtained by introducing mutations in one or more regions identified in the parent lipase.

### BRIEF DESCRIPTION OF THE FIGURES

FIG. 1 shows the alignment of lipases.

### SEQUENCE LISTINGS

SEQ ID NO: 1 shows the DNA sequence encoding lipase from Thermomyces lanoginosus.
SEQ ID NO: 2 shows the amino acid sequence of a lipase from Thermomyces lanoginosus.
SEQ ID NO: 3 shows the amino acid sequence of a lipase from Absidia reflexa.
SEQ ID NO: 4 shows the amino acid sequence of a lipase from Absidia corymbifera.
SEQ ID NO: 5 shows the amino acid sequence of a lipase from Rhizomucor miehei.
SEQ ID NO: 6 shows the amino acid sequence of a lipase from Rhizopus oryzae.
SEQ ID NO: 7 shows the amino acid sequence of a lipase from Aspergillus niger.
SEQ ID NO: 8 shows the amino acid sequence of a lipase from Aspergillus tubingensis.
SEQ ID NO: 9 shows the amino acid sequence of a lipase from Fusarium oxysporrum.
SEQ ID NO: 10 shows the amino acid sequence of a lipase from Fusarium heterosporum.
SEQ ID NO: 11 shows the amino acid sequence of a lipase from Aspergillus oryzae.
SEQ ID NO: 12 shows the amino acid sequence of a lipase from Penicillium camemberti.
SEQ ID NO: 13 shows the amino acid sequence of a lipase from Aspergillus foetidus.
SEQ ID NO: 14 shows the amino acid sequence of a lipase from Aspergillus niger.
SEQ ID NO: 15 shows the amino acid sequence of a lipase from Aspergillus oryzae.
SEQ ID NO: 16 shows the amino acid sequence of a lipase from Landerina penisapora.

### DETAILED DESCRIPTION OF THE INVENTION

Lipase Variants

Parent Lipase

The parent lipase may be a fungal lipase with an amino acid sequence having at least 50 % homology as defined in the section “Homology and alignment” to the sequence of the T. Lanoginosus lipase shown in SEQ IUD NO: 2.

The parent lipase may be a yeast polypeptide such as a Candida, Kluyveromyces, Pichia, Saccharomyces, Schizosaccharomyces, or Yarrowia polypeptide; or more preferably a filamentous fungal polypeptide such as an Acremonium, Aspergillus, Aureobasidium, Cryptococcus, Filobasidium, Fusarium, Humicola, Magnaporthe, Mucor, Myceliophthora, Neocallimastix, Neurospora, Paecilomyces, Penicillium, Piromyces, Schizophyllum, Talaromyces, Thermoascus, Thielavia, Tolypocladium, or Trichoderma polypeptide.

In a preferred aspect, the parent lipase is a Saccharomyces carlsbergensis, Saccharomyces cerevisiae, Saccharomyces diastaticus, Saccharomyces douglasii, Saccharomyces kluyveri, Saccharomyces norbensis, or Saccharomyces oviformis polypeptide having lipase activity.

In another preferred aspect, the parent lipase is an Aspergillus aculeatus, Aspergillus awamori, Aspergillus fumigatus, Aspergillus foetidus, Aspergillus japonicus, Aspergillus nidulans, Aspergillus niger, Aspergillus oryzae, Aspergillus turbigensis, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Thermomyces lanoginosus (synonym: Humicola lanuginose), Mucor miehei, Myceliophthora thermophila, Neurospora crassa, Penicillium purpurogenum, Trichoderma harzianum, Trichoderma koningii, Trichoderma longibrachiatum, Trichoderma reesei, or Trichoderma viride polypeptide.

In another preferred aspect, the parent lipase is a Thermomyces lipase.

In a more preferred aspect, the parent lipase is a Thermomyces Lanoginosus lipase. In an even more preferred embodiment the parent lipase is the lipase of SEQ ID NO: 2.

Identification of Regions and Substitutions.

The positions referred to in Region I through Region IV below are the positions of the amino acid residues in SEQ ID NO:2. To find the corresponding (or homologous) positions in a different lipase, the procedure described in “Homology and alignrmient” is used.

Substitutions in Region I

Region I consists of amino acid residues surrounding the N-terminal residue E1. In this region it is preferred to substitute an amino acid of the parent lipase with a more positive amino acid. Amino acid residues corresponding to the following positions are comprised by Region I: 2 to 11 and 223-239. The following positions are of particular interest: 4, 8, 11, 223, 227, 229, 231, 233, 234, 236. In particular the following substitutions have been identified: X4V, X227G, X231R and X233R.

In a preferred embodiment the parent lipase has at least 80%, such as 85% or 90%, such as at least 95% or 96% or 97% or 98% or 99%, identity to SEQ ID NO:2 . In a most preferred embodiment the parent lipase is identical to SEQ ID NO: 2.

Substitutions in Region II

Region II consists of amino acid residues in contact with substrate on one side of the acyl chain and one side of the alcohol part. In this region it is preferred to substitute an amino acid of the parent lipase with a more positive amino acid or with a less hydrophobic amino acid. Amino acid residues corresponding to the following positions are comprised by Region II: 202 to 211 and 249 to 269. The following positions are of particular interest : 202, 210, 211, 253, 254, 255, 256. In particular the following substitutions have been identified: X202G, X210K, X255Y/V and X256K/R.

In a preferred embodiment the parent lipase has at least 80%, such as 85% or 90%, such as at least 95% or 96% or 97% or 98% or 99%, identity to SEQ ID NO:2. In a most preferred embodiment the parent lipase is identical to SEQ ID NO: 2.

Substitutions in Region III

Region III consists of amino acid residues that form a flexible structure and thus allowing the substrate to get into the active site. In this region it is preferred to substitute an amino acid of the parent lipase with a more positive amino acid or a less hydrophobic amino acid. Amino acid residues corresponding to the following positions are comprised by Region imi: 82 to 102. The following positions are of particular interest: 83, 86, 87, 90, 91, 95, 96, 99. In particular the following substitutions have been identified: X83T, X86V and X90A/R.

In a preferred embodimennt the parent linpas has at least 80%, such as 85% or 90% such as at least 95% or 96% or 97% or 98% or 99%, identity to SEQ ID NO:2 . In a most preferred embodiment the parent lipase is identical to SEQ ID NO: 2.

Substitutions in Region IV

Region IV consists of amino acid residues that bind electrostatically to a surface. In this region it is preferred to substitute an amino acid of the parent lipase with a more positive amino acid. Amino acid residues corresponding to the following positions are comprised by Region IV: 27 and 54 to 62. The following positions are of particular interest: 27, 56, 57, 58, 60. In particular the following substitutions have been identified: X27R, X58N/AG/T/P and X60V/S/G/N/R/K/A/L.

In a preferred embodiment the parent lipase has at least 80%, such as 85% or 90%, such as at least 95% or 96% or 97% or 98% or 99%, identity to SEQ ID NO:2 . In a most preferred embodiment the parent lipase is identical to SEQ ID NO: 2.

Amino Acids at Other Positions

The parent lipase may optionally comprise substitution of other amino acids, particularly less than 10 or less than 5 such substitutions. Examples are substitutions corresponding to one or more of the positions 24, 46, 74, 81, 83, 127, 131, 137, 147, 150, 203, 206, 211, 263, 264, 265, 267 and 269 of the parent lipase. In a particular embodiment there is a substitution in at least one of the positions corresponding to position 81, 147, 150 and 249. In a preferred embodiment the at least one substitution is selected from the group consisting of X81Q/E, X147M/Y, X150G and X249R/I/L.

Further substitutions may, e.g., be made according to principles known in the art, e.g. substitutions described in WO 92/05249, WO 94/25577, WO 95/22615, WO 97/04079 and WO 97/07202.

Parent Lipase Variants

In one aspect, said variant, when compared to said parent, comprising a total of at least three substitutions, said substitutions being selected from one or more of the following groups of substitutions:

a) at least two substitutions in Region I,

b) at least one substitution in Region II,

c) at least one substitution in Region III, and/or

d) at least one substitution in Region IV.

The variant may comprise substitutions, compared to the variant's parent, corresponding to those substitutions listed below in Table 1.

TABLE 1
Some particular variants.

| Region I | Region II | Region III | Region IV | Outside regions |
|---|---|---|---|---|
| X4V + X227G + | X210K + | X83T + | X58A + | X150G |
| X231R + X233R | X256K | X86V | X60S | |
| X227 + X231R + | X256K | X86V | X58N + | X150G |
| X233R | | | X60S | |
| X231R + X233R | X255Y | | | |
| X231R + X233R | X202G | | | |
| X227G + X231R + | X256K | X86V | | |
| X233R | | | | |
| X4V + X231R + | | | X58N + | |
| X233R | | | X60S | |
| X231R + X233R | | X90R | X58N + | |
| | | | X60S | |
| X231R + X233R | X255V | X90A | | |
| X227G + X231R + | X256K | X86V | X58N + | X150G |
| X233R | | | X60S | |
| X231R + X233R | X211L | | X58N + | X147M |
| | | | X60S | |

In a further particular embodiment the parent lipase is identical to SEQ ID NO:2, and the variants of Table 1 will thus be:

TABLE 2
Some particular variants of SEQ ID NO:2

| Region I | Region II | Region III | Region IV | Outside regions |
|---|---|---|---|---|
| Q4V + L227G + | E210K + | S83T + | S58A + | A150G |
| T231R + N233R | P256K | I86V | V60S | |
| L227G + T231R + | P256K | I86V | S58N + | A150G |
| N233R | | | V60S | |
| T231R + N233R | I255Y | | | |
| T231R + N233R | I202G | | | |
| L227G + T231R + | P256K | I86V | | |
| N233R | | | | |
| QRV + T231R + | | | S58N + | |
| N233R | | | V60S | |
| T231R +N233R | | I90R | S58N + | |
| | | | V60S | |
| T231R + N233R | I255V | I90A | | |
| L227G + T231R + | P256K | I86V | S58N + | A150G |
| N233R | | | V60S | |
| T231R + N233R | F211L | | S58N + | L147M |
| | | | V60S | |

Nomenclature for Amino Acid Modifications

In describing lipase variants according to the invention, the following nomenclature is used for ease of reference: Original amino acid(s):position(s):substituted amino acid(s)

According to this nomenclature, for instance the substitution of glutamic acid for glycine in position 195 is shown as G195E. A deletion of glycine in the same position is shown as G195*, and insertion of an additional amino acid residue such as lysine is shown as G195GK. Where a specific lipase contains a “deletion” in comparison with other lipases and an insertion is made in such a position this is indicated as *36D for insertion of an aspartic acid in position 36. Multiple mutations are separated by pluses, i.e.: R170Y+G195E, representing mutations in positions 170 and 195 substituting tyrosine and glutamic acid for arginine and glycine, respectively.

X231 indicates the amino acid in a parent polypeptide corresponding to position 231, when applying the described alignment procedure. X231R indicates that the amino acid is replaced with R. For SEQ ID NO:2 X is T, and X231R thus indicates a substitution of T in position 231 with R. Where the amino acid in a position (e.g. 231) may be substituted by another amino acid selected from a group of amino acids, e.g. the group consisting of R and P and Y, this will be indicated by X231R/P/Y.

In all cases, the accepted IUPAC single letter or triple letter amino acid abbreviation is employed.

Amino Acid Grouping

In this specification, amino acids are classified as negatively charged, positively charged or electrically neutral according to their electric charge at pH 10. Thus, negative amino acids are E, D, C (cysteine) and Y, particularly E and D. Positive amino acids are R, K and H, particularly R and K. Neutral amino acids are CA A, V, L, I, P, F, W, S, T, M, N, Q and C when forming part of a disulfide bridge. A substitution with another amino acid in the same group (negative, positive or neutrall) is termed a conservative substitution.

The neutral amino acids may be divided into hydrophobic or non-polar (G. A, V, L, I, P, F, W and C as part of a disulfide bridge) and hydrophilic or polar (S, T, M, N, Q).

Amino Acid Identity

The relatedness between two amino acid sequences or between two nucleotide sequences is described by the parameter “identity”.

For purposes of the present invention, the alignment of two amino acid sequences is determined by using the Needle program from the EMBOSS package (http://emboss.org) version 2.8.0. The Needle program implements the global alignment algorithm described in Needleman, S. B. and Wunsch, C. D. (1970) J. Mol. Biol. 48, 443-453. The substitution matrix used is BLOSUM62, gap opening penalty is 10, and gap extension penalty is 0.5.

The degree of identity between an amino acid sequence of the present invention (“invention sequence”; e.g. amino acids 1 to 269 of SEQ ID NO:2) and a different amino acid sequence (“foreign sequence”) is calculated as the number of exact matches in an alignment of the two sequences, divided by the length of the “invention sequence” or the length of the “foreign sequence”, whichever is the shortest. The result is expressed in percent identity.

An exact match occurs when the “invention sequence” and the “foreign sequence” have identical amino acid residues in the same positions of the overlap. The length of a sequence is the number of amino acid residues in the sequence (e.g. the length of SEQ ID NO:2 is 269).

The parent lipase has an amino acid identity of at least 50% with the T Lanoginosus lipase (SEQ ID NO: 2), particularly at least 55%, at least 60%, at least 75%, at least 85% , at least 90%, more than 95% or more than 98%. In a particular embodiment the parent lipase is identical to the T Lanoginosus lipase (SEQ ID NO:2).

The above procedure may be used for calculation of identity as well as homology and for alignment. In the context of the present invention homology and alignment has been calculated as described below.

Homology and Alignment

For purposes of the present invention, the degree of homology may be suitably determined by means of computer programs known in the art, such as GAP provided in the GCG program package (Program Manual for the Wisconsin Package, Version 8, August 1994, Genetics Computer Group, 575 Science Drive, Madison, Wis., USA 53711) (Needleman, S. B. and Wunsch, C. D., (1970), Journal of Molecular Biology, 48, 443-45), using GAP with the following settings for polypeptide sequence comparison: GAP creation penalty of 3.0 and GAP extension penalty of 0.1.

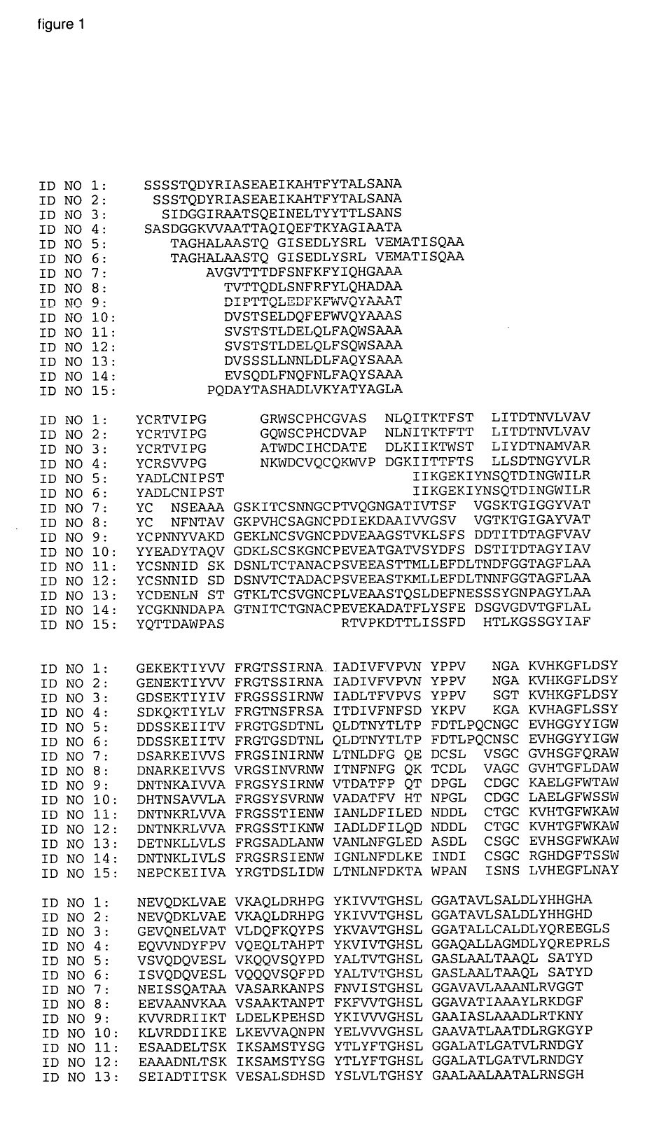

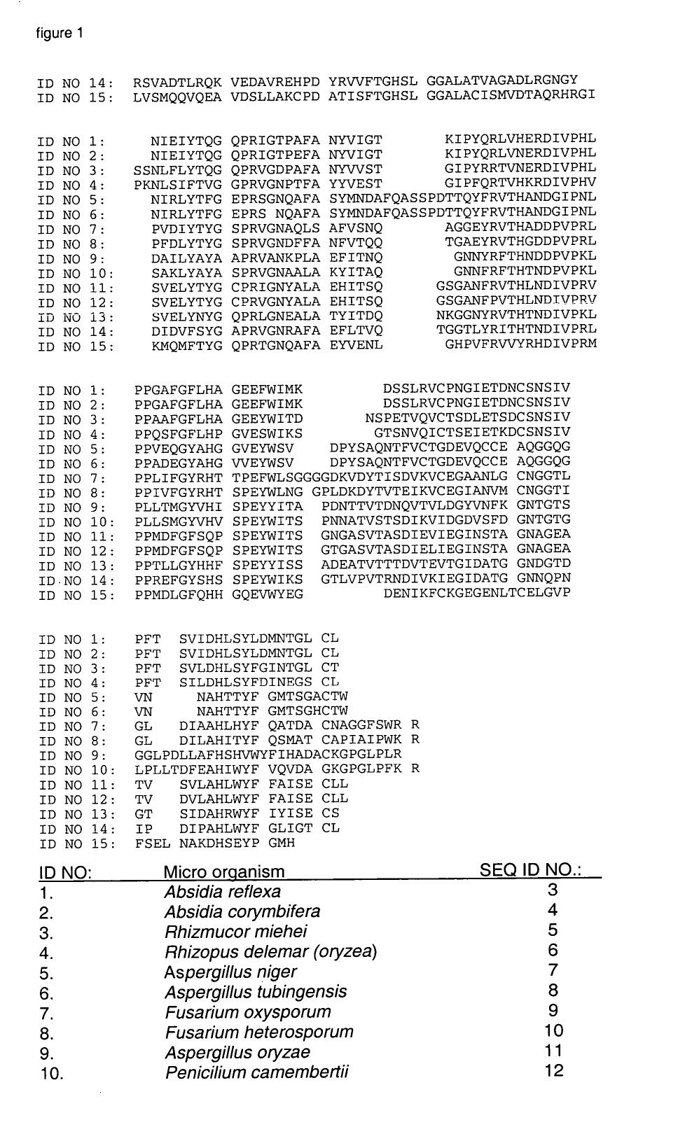

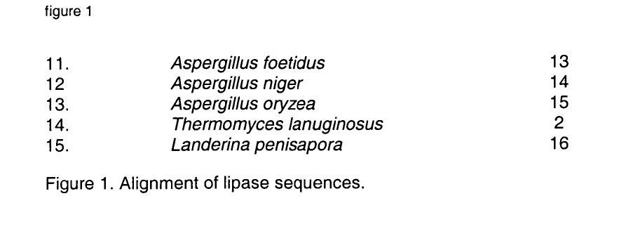

In the present invention, corresponding (or homologous) positions in the lipase sequences of Absidia reflexa, Absidia corymbefera, Rhizmucor miehei, Rhizopus delemar, Aspergillus niger, Aspergillus tubigensis, Fusarium oxysporum, Fusarium heterosporum, Aspergillus oryzea, Penicilium camembertii, Aspergillus foetidus, Aspergillus niger, Thermomyces lanoginosus (synonym: Humicola lanuginose) and Landerina penisapora are defined by the alignment shown in FIG. 1.

To find the homologous positions in lipase sequences not shown in the alignment, the sequence of interest is aligned to the sequences shown in FIG. 1. The new sequence is aligned to the present alignment in FIG. 1 by using the GAP alignment to the most homologous sequence found by the GAP program. GAP is provided in the GCG program package (Program Manual for the Wisconsin Package, Version 8, August 1994, Genetics Computer Group, 575 Science Drive, Madison, Wis., USA 53711) (Needleman, S. B. and Wunsch, C. D., (1970), Journal of Molecular Biology, 48, 443-45). The following settings are used for polypeptide sequence comparison: GAP creation penalty of 3.0 and GAP extension penalty of 0.1.

The parent lipase has a homology of at least 50% with the T. Lanoginosus lipase (SEQ ID NO: 2), particularly at least 55%, at least 60%, at least 75%, at least 85% , at least 90%, more than 95% or more than 98%. In a particular embodiment the parent lipase is identical to the T. Lanoginosus lipase (SEQ ID NO:2).

Hybridization

The present invention also relates to isolated polypeptides having lipase activity which are encoded by polynucleotides which hybridize under very low stringency conditions, preferably low stringency conditions, more preferably medium stringency conditions, more preferably medium-high stringency conditions, even more preferably high stringency conditions, and most preferably very high stringency conditions with (i) nucleotides 178 to 660 of SEQ ID NO: 1, (ii) the cDNA sequence contained in nucleotides 178 to 660 of SEQ ID NO: 1, (iii) a subsequence of (i) or (ii), or (iv) a complementary strand of (i), (ii), or (iii) (J. Sambrook, E. F. Fritsch, and T. Maniatus, 1989, Molecular Cloning, A Laboratory Manual, 2d edition, Cold Spring Harbor, New York). A subsequence of SEQ ID NO: 1 contains at least 100 contiguous nucleotides or preferably at least 200 contiguous nucleotides. Moreover, the subsequence may encode a polypeptide fragment which has lipase activity.

For long probes of at least 100 nucleotides in length, very low to very high stringency conditions are defined as prehybridization and hybridization at 42° C. in 5×SSPE, 0.3% SDS, 200 ug/ml sheared and denatured salmon sperm DNA, and either 25% formamide for very low and low stringencies, 35% formamide for medium and medium-high stringencies, or 50% formamide for high and very high stringencies, following standard Southern blotting procedures for 12 to 24 hours optimally.

For long probes of at least 100 nucleotides in length, the carrier material is finally washed three times each for 15 minutes using 2×SSC, 0.2% SDS preferably at least at 45° C. (very low stringency), more preferably at least at 50° C. (low stringency), more preferably at least at 55° C. (medium stringency), more preferably at least at 60° C. (medium-high stringency), even more preferably at least at 65° C. (high stringency), and most preferably at least at 70° C. (very high stringency).

DNA Sequence, Expression Vector, Host Cell, Production of Lipase

The invention provides a DNA sequence encoding the lipase of the invention, an expression vector harboring the DNA sequence, and a transformed host cell containing the DNA sequence or the expression vector. These may be obtained by methods known in the art.

The invention also provides a method of producing the lipase by culturing the transformed host cell under conditions conducive for the production of the lipase and recovering the lipase from the resulting broth. The method may be practiced according to principles known in the art.

Lipase Activity

Lipase Activity on Tributyrin at Neutral pH (LU)

A substrate for lipase is prepared by emulsifying tributyrin (glycerin tributyrate) using gum Arabic as emulsifier. The hydrolysis of tributyrin at 30° C. at pH 7 or 9 is followed in a pH-stat titration experiment. One unit of lipase activity (1 LU) equals the amount of enzyme capable of releasing 1 micro mol butyric acid/min at pH 7.

Benefit Risk

The Benefit Risk factor describing the performance compared to the reduced risk for odour smell is defined as: BR=RP_{avg}/R. Lipase variants described herein may have BRs greater than 1, greater than 1.1, or even greater than 1 to about 1000.

Average Relative Performance

The procedure for calculating average relative performance (RPavg) is found in Example 5 of the present specification. Lipase variants described herein may have (RPavg) of at least 0.8, at least 1.1, at least 1.5, or even at least 2 to about 1000.

Detergent Ingredients

As used herein detergent compositions include articles and cleaning and treatment compositions. As used herein, the term “cleaning and/or treatment composition” includes, unless otherwise indicated, tablet, granular or powder-form all-purpose or “heavy-duty” washing agents, especially laundry detergents; liquid, gel or paste-form all-purpose washing agents, especially the so-called heavy-duty liquid types; liquid fine-fabric detergents; hand dishwashing agents or light duty dishwashing agents, especially those of the high-foaming type; machine dishwashing agents, including the various tablet, granular, liquid and rinse-aid types for household and institutional use. The compositions can also be in unit dose packages, including those known in the art and those that are water soluble, water insoluble and/or water permeable.

The detergent composition of the present invention can comprise one or more lipase variant(s) of the present invention. In addition to the lipase variant(s), the detergent composition will further comprise a detergent ingredient. The non-limiting list of detergent ingredients illustrated hereinafter are suitable for use in the instant compositions and may be desirably incorporated in certain embodiments of the invention, for example to assist or enhance cleaning performance, for treatment of the substrate to be cleaned, or to modify the aesthetics of the cleaning composition as is the case with colorants, dyes or the like. The precise nature of these additional components, and levels of incorporation thereof, will depend on the physical form of the composition and the nature of the cleaning operation for which it is to be used. Suitable detergent ingredients include, but are not limited to, surfactants, builders, chelating agents, dye transfer inhibiting agents, dispersants, enzymes, and enzyme stabilizers, bleach activators, hydrogen peroxide, sources of hydrogen peroxide, preformed peracids, polymeric dispersing agents, brighteners, suds suppressors, dyes, anti-corrosion agents, tarnish inhibitors, perfumes, fabric softeners, carriers, hydrotropes, processing aids, solvents and/or pigments.

Typical detergents would comprise by weight any combination of the following ingredients: 5-30% surfactant, preferably anionic surfactants such as linear alkylbenzenesulfonate and alcohol ethoxysulfate; 0.005-0.1% protease active protein, wherein the protease is preferably selected from Coronasem™, FN4 FNA, or Savinase™, 0.001-0.1% amylase active protein, wherein the amylase is preferably selected from Termamyl™ Natalasem™, Stainzyme™ and Purastar™ and 0.1-3% chelants, preferably diethylene triamine pentaacetic acid. For granular and tablet products, such typical detergents would additionally comprise by weight: 5-20% bleach, preferably sodium percarbonate; 1-4% bleach activator, preferably TAED and/or 0-30% builder, preferably 5-30%, more preferably less than 10% builder, such as the aluminosilicate Zeolite A and/or tripolyphosphate.

Bleaching Agents—The detergent compositions of the present invention may comprise one or more bleaching agents.

In general, when a bleaching agent is used, the compositions of the present invention may comprise from about 0.1% to about 50% or even from about 0.1% to about 25% bleaching agent by weight of the subject cleaning composition. Examples of suitable bleaching agents include:

(1) sources of hydrogen peroxide, for example, inorganic perhydrate salts, including alkali metal salts such as sodium salts of perborate (usually mono- or tetra-hydrate), percarbonate, persulphate, perphosphate, persilicate salts and mixtures thereof. In one aspect of the invention the inorganic perhydrate salts are selected from the group consisting of sodium salts of perborate, percarbonate and mixtures thereof. soaps; and

(2) bleach activators having R—(C═O)—L wherein R is an alkyl group, optionally branched, having, when the bleach activator is hydrophobic, from 6 to 14 carbon atoms, or from 8 to 12 carbon atoms and, when the bleach activator is hydrophilic, less than 6 carbon atoms or even less than 4 carbon atoms; and L is leaving group. Examples of suitable leaving groups are benzoic acid and derivatives thereof—especially benzene sulphonate. Suitable bleach activators include dodecanoyl oxybenzene sulphonate, decanoyl oxybenzene sulphonate, decanoyl oxybenzoic acid or salts thereof, 3,5,5-trimethyl hexanoyloxybenzene sulphonate, tetraacetyl ethylene diamine (TAED) and nonanoyloxybenzene sulphonate (NOBS). Suitable bleach activators are also disclosed in WO 98/17767. While any suitable bleach activator may be employed, in one aspect of the invention the subject cleaning composition may comprise NOBS, TAED or mixtures thereof.

(3) Pre-formed peracids.

When present, the peracid and/or bleach activator is generally present in the composition in an amount of from about 0.1 to about 60 wt %, from about 0.5 to about 40 wt % or even from about 0.6 to about 10 wt % based on the composition. One or more hydrophobic precursors thereof may be used in combination with one or more hydrophilic peracid or precursor thereof.

The amounts of hydrogen peroxide source and peracid or bleach activator may be selected such that the molar ratio of available oxygen (from the peroxide source) to peracid is from 1:1 to 35:1, or even 2:1 to 10:1.

Surfactants—The detergent compositions according to the present invention may comprise a surfactant or surfactant system wherein the surfactant can be selected from nonionic surfactants, anionic surfactants, cationic surfactants, ampholytic surfactants, zwitterionic surfactants, semi-polar nonionic surfactants and mixtures thereof. When present, surfactant is typically present at a level of from about 0.1% to about 60%, from about 0.1% to about 40%, from about 0.1% to about 12%, from about 1% to about 50% or even from about 5% to about 40% by weight of the subject composition.

When included therein the detergent will usually contain from about 1% to about 40% of an anionic surfactant such as linear alkylbenzenesulfonate, alpha-olefinsulfonate, alkyl sulfate (fatty alcohol sulfate), alcohol ethoxysulfate, secondary alkanesulfonate, alpha-sulfo fatty acid methyl ester, alkyl- or alkenylsuccinic acid or soap.

The detergent may optionally contain from about 0.2% to about 40% of a non-ionic surfactant such as alcohol ethoxylate, nonylphenol ethoxylate, alkylpolyglycoside, alkyldimethylamineoxide, ethoxylated fatty acid monoethanolamide, fatty acid monoethanolamide, polyhydroxy alkyl fatty acid amide, or N-acyl N-alkyl derivatives of glucosamine (“glucamides”).

Builders—The detergent compositions of the present invention may comprise one or more detergent builders or builder systems. When a builder is used, the subject composition will typically comprise at least about 1%, from about 5% to about 60% or even from about 10% to about 40% builder by weight of the subject composition. Builders include, but are not limited to, the alkali metal, ammonium and alkanolammonium salts of polyphosphates, alkali metal silicates or layered silicates, alkaline earth and alkali metal carbonates, aluminosilicate builders and the various alkali metal, ammonium and substituted ammonium salts of polyacetic acids such as ethylenediamine tetraacetic acid and nitrilotriacetic acid, as well as polycarboxylates such as mellitic acid, succinic acid, citric acid, oxydisuccinic acid, polymaleic acid, benzene 1,3,5-tricarboxylic acid, carboxymethyloxysuccinic acid, and soluble salts thereof.

Chelating Agents—The detergent compositions herein may contain a chelating agent. Suitable chelating agents include copper, iron and/or manganese chelating agents and mixtures thereof. When a chelating agent is used, the subject composition may comprise from about 0.005% to about 15% or even from about 3.0% to about 10% chelating agent by weight of the Subhiert conmposition.

Brighteners—The detergent compositions of the present invention can also contain additional components that may alter appearance of articles being cleaned, such as fluorescent brighteners. These brighteners absorb in the UV-range and emnit in the visible. Suitable fluorescent brightener levels include lower levels of from about 0.01, from about 0.05, from about 0.1 or even from about 0.2 wt % to upper levels of 0.5 or even 0.75 wt %.

Dispersants—The compositions of the present invention can also contain dispersants. Suitable water-soluble organic materials include the homo- or co-polymeric acids or their salts, in which the polycarboxylic acid comprises at least two carboxyl radicals separated from each other by not more than two carbon atoms.

Enzymes—In addition to the lipase variant(s) of the present invention the detergent composition can comprise one or more further enzymes which provide cleaning performance and/or fabric care benefits such as a protease, another lipase, a cutinase, an amylase, a carbohydrase, a cellulase, a pectinase, a mannanase, an arabinase, a galactanase, a xylanase, an oxidase, e.g., a laccase, and/or a peroxidase.

In general the properties of the chosen enzyme(s) should be compatible with the selected detergent, (i.e. pH-optimum, compatibility with other enzymatic and non-enzymatic ingredients, etc.), and the enzyme(s) should be present in effective amounts.

Suitable proteases include those of animal, vegetable or microbial origin. Microbial origin is preferred. Chemically modified or protein engineered mutants are included. The protease may be a serine protease or a metallo protease, preferably an alkaline microbial protease or a trypsin-like protease. Examples of alkaline proteases are subtilisins, especially those derived from Bacillus, e.g., subtilisin Novo, subtilisin Carlsberg, subtilisin 309, subtilisin 147 and subtilisin 168 (described in WO 89/06279), SEQ ID no 4 and SEQ ID no 7 in WO 05/103244. Other suitable serin proteases include those from Micrococcineae spp especially Cellulonas spp and variants thereof as disclosured in WO2005052146. Examples of trypsin-like proteases are trypsin (e.g. of porcine or bovine origin) and the Fusarium protease described in WO 89/06270 and WO 94/25583.

Examples of useful proteases are the variants described in WO 92/19729, WO 98/20115, WO 98/20116, and WO 98/34946, especially the variants with substitutions in one or more of the following positions: 27, 36, 57, 68, 76, 87, 97, 101, 104, 106, 120, 123, 167, 170, 194, 206, 218, 222; 224, 235, 245, 252 and 274, and amongst other variants with the following mutations: (K27R, V104Y, N123S, T124A), (N76D, S103A, V1041), or (S101G, S103A, V104I, G159D, A232V, Q236H, Q245R, N248D, N252K). Other examples of useful proteases are the variants described in WO 05/052146 especially the variants with substitutions in one or more of the following positions: 14, 16, 35, 65, 75, 76, 79, 123, 127, 159 and 179

Preferred commercially available protease enzymes include Alcalase™, Savinase™, Primase™, Duralase™, Esperase™, Coronase™, Polarzyme™ and Kannase™ (Novozymes A/S), Maxatase™, Maxacal™, Maxapem™, Properase™, Purafect™, Purafect Prime™, Purafect OxP™, FN2, FN3 and FN4 (Genencor International Inc.).

Lipases include those of bacterial or fungal origin. Chemically modified or protein engineered mutants are included. Examples of useful lipases include lipases from Humicola (synonym Thermomyces), e.g. from H. lanuginosa (synonymous T. Lanoginosus) as described in EP 258 068 and EP 305 216 or from H. insolens as described in WO 96/13580, a Pseudomonas lipase, e.g. from P. alcaligenes or P. pseudoalcaligenes (EP 218 272), P. cepacia (EP 331 376), P. stutzeri (GB 1,372,034), P. fluorescens, Pseudomonas sp. strain SD 705 (WO 95/06720 and WO 96/27002), P. wisconsinensis (WO 96/12012), a Bacillus lipase, e.g. from B. subtilis (Dartois et al. (1993), Biochemica et Biophysica Acta, 1131, 253-360), B. stearothermophilus (JP 64/744992) or B. pumilus (WO 91/16422).

Other examples are lipase variants such as those described in WO 92/05249, WO 94/01541, EP 407 225, EP 260 105, WO 95/35381, WO 96/00292, WO 95/30744, WO 94/25578, WO 95/14783, WO 95/22615, WO 97/04079 and WO 97/07202.

Other commercially available lipase enzymes include Lipolase™, Lipolase Ultra™ and Lipex™ (Novozymes A/S).

Suitable amylases (α and/or β) include those of bacterial or fungal origin. Chemically modified or protein engineered mutants are included. Amylases include, for example, α-amylases obtained from Bacillus, e.g. a special strain of B. licheniformis, described in more detail in GB 1,296,839.

Examples of useful amylases are the variants described in WO 94/02597, WO 94/18314, WO 96/23873, and WO 97/43424, especially the variants with substitutions in one or more of the following positions: 15, 23, 105, 106, 124, 128, 133, 154, 156, 181, 188, 190, 197, 202, 208, 209, 243, 264, 304, 305, 391, 408, and 444.

Commercially available amylases are Duramyl™, Termamyl™, Stainzyme™, Stainzyme Ultra™, Fungamyl™ and BAN™ (Novozymes A/S), Rapidase™ and Purastar™ (from Genencor International Inc.).

Suitable cellulases include those of bacterial or fungal origin. Chemically modified or protein engineered mutants are included. Suitable cellulases include cellulases from the genera Bacillus, Pseudomonas, Humicola, Fusarium, Thielavia, Acremonium, e.g. the fungal cellulases produced from Humicola insolens, Myceliophthora thermophila and Fusarium oxysporum disclosed in U.S. Pat. Nos. 4,435,307, 5,648,263, 5,691,178, 5,776,757 and WO 89/09259.

Especially suitable cellulases are the alkaline or neutral cellulases having colour care benefits. Examples of such cellulases are cellulases described in EP 0 495 257, EP 0 531 372, WO 96/11262, WO 96/29397, WO 98/08940. Other examples are cellulase variants such as those described in WO 94/07998, EP 0 531 315, U.S. Pat Nos. 5,457,046, 5,686,593, 5,763,254, WO 95/24471, WO 98/12307 and PCT/DK98/00299.

Commercially available cellulases include Renozyme™, Celluclean™, Endolase,™ Celluzyme™, and Carezyme™ (Novozymes A/S), Clazinase™, and Puradax HA™ (Genencor International Inc.), and KAC-500(B)™ (Kao Corporation). Peroxidases/Oxidases:

Suitable peroxidases/oxidases include those of plant, bacterial or fungal origin. Chemically modified or protein engineered mutants are included. Examples of useful peroxidases include peroxidases from Coprinus, e.g. from C. cinereus, and variants thereof as those described in WO 93/24618, WO 95/10602, and WO 98/15257.

Commercially available peroxidases include Guardzyme™ (Novozymes A/S). When present in a cleaning composition, the aforementioned enzymes may be present at levels from about 0.00001% to about 2%, from about 0.0001% to about 1% or even from about 0.001% to about 0.5% enzyme protein by weight of the composition.

Enzyme Stabilizers—Enzymes for use in detergents can be stabilized by various techniques. The enzymes employed herein can be stabilized by the presence of water-soluble sources of calcium and/or magnesium ions in the finished compositions that provide such ions to the enzymes. Further conventional stabilizing agents, e.g., a polyol such as propylene glycol or glycerol, a sugar or sugar alcohol, lactic acid, boric acid, or a boric acid derivative, e.g., an aromatic borate ester, or a phenyl boronic acid derivative such as 4-formylphenyl boronic acid, may also be used and the composition may be formulated as described in e.g. WO 92/19709 and WO 92/19708.

Solvents—Suitable solvents include water and other solvents such as lipophilic fluids. Examples of suitable lipophilic fluids include siloxanes, other silicones, hydrocarbons, glycol ethers, glycerine derivatives such as glycerine ethers, perfluorinated amines, pefluorinated and hydrofluoroether solvents, low-volatility nonfluorinated organic solvents, diol solvents, other environmentally-friendly solvents and mixtures thereof.

Washing Method

The present invention includes a method for cleaning and/or treating a situs inter alia a surface or fabric. Such method includes the steps of contacting an embodiment of Applicants' cleaning composition, in neat form or diluted in a wash liquor, with at least a portion of a surface or fabric then optionally rinsing such surface or fabric. The surface or fabric may be subjected to a washing step prior to the aforementioned rinsing step. For purposes of the present invention, washing includes but is not limited to, scrubbing, and mechanical agitation. As will be appreciated by one skilled in the art, the cleaning compositions of the present invention are ideally suited for use in laundry applications. Accordingly, the present invention includes a method for laundering a fabric. The method comprises the steps of contacting a fabric to be laundered with a said cleaning laundry solution comprising at least one embodiment of Applicants' cleaning composition, cleaning additive or mixture thereof. The fabric may comprise most any fabric capable of being laundered in normal consumer use conditions. The solution preferably has a pH of from about 8 to about 10.5. The compositions may be employed at concentrations of from about 100 ppm, preferably 500 ppm to about 15,000 ppm in solution. The water temperatures typically range from about 5° C. to about 90° C. The invention may be particularly beneficial at low water temperatures such as below 30° C. or below 25 or 20° C. The water to fabric ratio is typically from about 1:1 to about 30:1.

Lipase Variants Examples

Chemicals used as buffers and substrates were commercial products of at least reagent grade.
Media and Solutions: LAS (Surfac PS™) and Zeolite A (Wessalith P™). Other ingredients used are standard laboratory reagents.
Materials: EMPA221 from EMPA St. Gallen, Lerchfeldstrasse 5, CH-9014 St. Gallen, Switzerland

### EXAMPLE 1

Production of Enzyme

A plasmid containing the gene encoding the lipase is constructed and transformed into a suitable host cell using standard methods of the art.

Fermentation is carried out as a fed-batch fermentation using a constant medium temperature of 34° C. and a start volume of 1.2 liter. The initial pH of the medium is set to 6.5. Once the pH has increased to 7.0 this value is maintained through addition of 10% H3PO4. The level of dissolved oxygen in the medium is controlled by varying the agitation rate and using a fixed aeration rate of 1.0 liter air per liter medium per minute. The feed addition rate is maintained at a constant level during the entire fed-batch phase.

The batch medium contained maltose syrup as carbon source, urea and yeast extract as nitrogen source and a mixture of trace metals and salts. The feed added continuously during the fed-batch phase contains maltose syrup as carbon source whereas yeast extract and urea is added in order to assure a sufficient supply of nitrogen.

Purification of the lipase may be done by use of standard methods known in the art, e.g. by filtering the fermentation supernatant and subsequent hydrophobic chromatography and anion exchange, e.g. as described in EP 0 851 913, Example 3.

### EXAMPLE 2

AMSA—Automated Mechanical Stress Assay—for Calculation of Relative Performance (RP).

The enzyme variants of the present application are tested using the Automatic Mechanical Stress Assay (AMSA). With the AMSA test the wash performance of a large quantity of small volume enzyme-detergent solutions can be examined. The AMSA plate has a number of slots for test solutions and a lid firmly squeezing the textile swatch to be washed against all the slot openings. During the washing time, the plate, test solutions, textile and lid are vigorously shaken to bring the test solution in contact with the textile and apply mechanical stress. For further description see WO 02/42740 especially the paragraph “Special method embodiments” at page 23-24. The containers, which contain the detergent test solution, consist of cylindrical holes (6 mm diameter, 10 mm depth) in a metal plate. The stained fabric (test material) lies on the top of the metal plate and is used as a lid and seal on the containers. Another metal plate lies on the top of the stained fabric to avoid any spillage from each container. The two metal plates together with the stained fabric are vibrated up and down at a frequency of 30 Hz with an amplitude of 2 mm.

The assay is conducted under the experimental conditions specified below:

TABLE 3

| | |
|---|---|
| Test solution | 0.5 g/l LAS |
| | 0.52 g/l Na2CO3 |
| | 1.07 g/l Zeolite A |
| | 0.52 g/l Tri sodium Citrate |
| Test solution volume | 160 micro 1 |
| pH | As is (≈9.9) |
| Wast time | 20 minutes |
| Temperature | 30° C. |
| Water hardness | 15° dH |
| | Ratio of Ca²⁺/Mg²⁺/NaHCO₃:4:1:7.5 |
| Enzyme concentration in | 0.125, 0.25, 0.50, 1.0 mg enzyme protein/liter |
| test solution | (mg ep/1) |
| Drying | Performance: After washing the textile pieces is |
| | immediately flushed in tap water and air-dried |
| | at 85° C. in 5 min |
| | Odor: After washing the textile pieces is |
| | immediately flushed in tap water and dried at |
| | room temperature (20° C.) for 2 hours |
| Test material | Cream turmeric swatch as described below |
| | (EMPA221 used as cotton textile) |

Cream-turmeric swatches were prepared by mixing 5 g of turmeric (Santa Maria, Denmark) with 100 g cream (38% fat, Arla, Denmark) at 50° C., the mixture is left at this temperature for about 20 minutes and filtered (50° C.) to remove any undissolved particles. The mixture is cooled to 20° C.) woven cotton swatches, EMPA221, were immersed in the cream-turmeric mixture and afterwards allowed to dry at room temperature over night and frozen until use. The preparation of cream-turmeric swatches is disclosed in the patent application PA 2005 00775, filed 27 May 2005.

The performance of the enzyme variant is measured as the brightness of the colour of the textile samples washed with that specific enzyme variant. Brightness can also be expressed as the intensity of the light reflected from the textile sample when luminated with white light. When the textile is stained the intensity of the reflected light is lower, than that of a clean textile. Therefore the intensity of the reflector light can be to measure wash performance of an enzyme variant.

Color measurements are made with a professional flatbed scanner (PFU DL2400pro), which is used to capture an image of the washed textile samples. The scans are made with a resolution of 200 dpi and with an output color depth of 24 bits. In order to get accurate results, the scanner is frequently calibrated with a Kodak reflective IT8 target.

To extract a value for the light intensity from the scanned images, a special designed software application is used (Novozymes Color Vector Analyzer). The program retrieves the 24 bit pixel values from the image and converts them into values for red, green and blue (RGB). The intensity value (Int) is calculated by adding the RGB values together as vectors and then taking the length of the resulting vector:
Int=\sqrt{r²+g²+b²}.

The wash performance (P) of the variants is calculated in accordance with the formula:
P=Int(v)−Int(r) where
Int(v) is the light intensity value of textile surface washed with the tested enzyme and Int(r) is the light intensity value of textile surface washed without the tested enzyme.

A relative performance score is given as the result of the AMSA wash in accordance with the definition: Relative Performance scores (RP) are summing up the performances (P) of the tested enzyme variants against the reference enzyme: RP=P(test enzyme)/P(reference enzyme). RPavg indicates the average relative performance compared to the reference enzyme at all four enzyme concentrations (0.125, 0.25, 0.5, 1.0 mg ep/l)
RPavg=avg(RP(0.125),RP(0.25)RP(0.5),RP(1.0))
A variant is considered to exhibit improved wash performance, if it performs better than the reference. In the context of the present invention the reference enzyme is the lipase of SEQ ID NO:2 with the substitutions T231R+N233R.

### EXAMPLE 3

GC—Gas Chromatograph—for Calculation of Risk Factor.

The butyric acid release from the lipase washed swatches were measured by Solid Phase Micro Extraction Gas Chromatography (SPME-GC) using the following method. Four textile pieces (5 mm in diameter), washed in the specified solution in Table 3 containing 1 mg/l lipase, were transferred to a Gas Chromatograph (GC) vial. The samples were analysed on a Varian 3800 GC equipped with a Stabilwax-DA w/Integra-Guard column (30 m, 0.32 mm ID and 0.25 micro-m df) and a Carboxen PDMS SPME fibre (75 micro-m). Each sample is preincubated for 10 min at 40° C. followed by 20 min sampling with the SPME fibre in the head-space over the textile pieces. The sample is subsequently injected onto the column (injector temperature=250° C.). Column flow=2 ml Helium/min. Column oven temperature gradient: 0 min=40° C., 2 min=40° C., 22 min=240° C., 32 min=240° C. The butyric acid is detected by FID detection and the amount of butyric acid is calculated based on a butyric acid standard curve.

The Risk Performance Odour, R, of a lipase variant is the ratio between the amount of released butyric acid from the lipase variant washed swatch and the amount of released butyric acid from a swatch washed with the lipase of SEQ ID NO: 2 with the substitutions T231R+N233R (reference enzyme), after both values have been corrected for the amount of released butyric acid from a non-lipase washed swatch. The risk (R) of the variants is calculated in accordance with the below formula:
Odour=measured in micro g butyric acid developed at 1 mg enzyme protein/1 corrected for blank
α_{test enzyme}=Odour_{test enzyme}−Blank
α_{reference enzyme}=Odour_{reference enzyme}−Blank
R=α_{test enzyme}/α_{reference enzyme}
A variant is considered to exhibit reduced odor compared to the reference, if the R factor is lower than 1.

### EXAMPLE 4

Activity (LU) Relative to Absorbance at 280 nm

The activity of a lipase relative to the absorbance at 280 nm is determined by the following assay

LU/A280:

The activity of the lipase is determined as described above in the section Lipase activity. The absorbance of the lipase at 280 nm is measured (A280) and the ratio LU/A280 is calculated. The relative LU/A280 is calculated as the LU/A280 of the variant divided by the LU/A280 of a reference enzyme. In the context of the present invention the reference enzyme is the lipase of SEQ ID NO:2 with the substitutions T231R+N233R.

### EXAMPLE 5

BR—Benefit Risk

The Benefit Risk factor describing the performance compared to the reduced risk for odour smell is thus defined as: BR=RP_{avg}/R

A variant is considered to exhibit improved wash performance and reduced odor, if the BR factor er than 1.

Applying the above methods the following results were obtained:

TABLE 4

| | | Average | | |
|---|---|---|---|---|
| | Mutations in | RP | | |
| Variant | SEQ ID NO: 2 | (RP_{avg}) | BR | LU/A280 |
| 1 | I202G + T231R + | 0.84 | 1.41 | not |
| | N233R | | | determined |
| 2 | I86V + L227G + | 1.08 | 1.52 | 1700 |
| | T231R + N233R + | | | |
| | P256K | | | |
| 3 | Q4V + S58N + | 0.87 | 1.73 | 1950 |
| | V60S + T231R + | | | |
| | N233R | | | |
| 4 | S58N + V60S + | 1.06 | 1.27 | 2250 |
| | I90R + T231R + | | | |
| | N233R | | | |
| 5 | I255Y + T231R + | 1.19 | 1.17 | 3600 |
| | N233R | | | |
| 6 | I90A + T231R + | 1.13 | 1.14 | 2700 |
| | N233R + I255V | | | |
| Reference | T231R + N233R | 1.00 | 1.00 | 3650 |
| 7 | G91A + E99K + | 0.43 | not | 850 |
| | T231R + N233R + | | determined | |
| | Q249R + 270H + | | | |
| | 271T + 272P + 273S + | | | |
| | 274S + 275G + | | | |
| | 276R + 277G + | | | |
| | 278G + 279H + | | | |
| | 280R | | | |
| 8 | G91A + E99K + | 0.13 | not | 500 |
| | T231R, N233R + | | determined | |
| | Q249R + 270H + | | | |
| | 271T + 272P + | | | |
| | 273S + 274S + | | | |
| | 275G + 276R + | | | |
| | 277G + 278G | | | |

The reference lipase and variants 7 and 8 in Table 4 are described in WO 2000/060063.

### DETERGENT EXAMPLES

Abbreviated component identifications for the examples are as follows:
LAS Sodium linear C₁₁₋₁₃ alkyl benzene sulphonate.
CxyAS Sodium C₁ₓ-C_{1y} alkyl sulfate.
CxyEzS C₁ₓ-C_{1y} sodium alkyl sulfate condensed with an average of z moles of ethylene oxide.
CxyEy C₁ₓ-C_{1y} alcohol with an average of ethoxylation of z
QAS R₂.N+(CH₃)₂(C₂H₄OH) with R₂=C₁₀-C₁₂
Silicate Amorphous Sodium Silicate (SiO₂:Na₂O ratio=1.6-3.2:1).
Zeolite A Hydrated Sodium Aluminosilicate of formula Na₁₂(AlO₂SiO₂)₁₂. 27H₂O having a primary particle size in the range from 0.1 to 10 micrometers (Weight expressed on an anhydrous basis).
(Na-)SKS-6 Crystalline layered silicate of formula δ-Na₂Si₂O5.
Citrate Tri-sodium citrate dihydrate.
Citric Anhydrous citric acid.
Carbonate Anhydrous sodium carbonate.
Sulphate Anhydrous sodium sulphate.
MA/AA Random copolymer of 4:1 acrylate/maleate, average molecular weight about 70,000-80,000.
AA polymer Sodium polyacrylate polymer of average molecular weight 4,500.
PB1/PB4 Anhydrous sodium perborate monohydrate/tetrahydrate.
PC3 Anhydrous sodium percarbonate [2.74 Na₂CO₃.3H₂O₂]
TAED Tetraacetyl ethylene diamine.
NOBS Nonanoyloxybenzene sulfonate in the form of the sodium salt.
DTPA Diethylene triamine pentaacetic acid.
HEDP Hydroxyethane di phosphonate
EDDS Na salt of Ethylenediamine-N,N′-disuccinic acid, (S,S) isomer
STPP Sodium tripolyphosphate
Protease Proteolytic enzyme sold under the tradename Savinase® Alcalase®, Everlase®, Coronase®, Polarzyme®, by Novozymes A/S, Properase®, Purafect®, Purafect MA® and Purafect Ox® sold by Genencor and proteases described in patents WO 91/06637 and/or WO 95/10591 and/or EP 0 251 446 such as FNA, FN3 and/or FN4.
Amylase Amylolytic enzyme sold under the tradename Purastar®, Purafect Oxam® sold by Genencor; Termamyl®, Fungamyl® Duramyl®, Stainzyme® and Natalase® sold by Novozymes A/S.
Lipase Any lipase variant 1 to 5 described in example 5 Table 4, and combinations thereof.
Mannanase Mannaway® sold by Novozymes
CMC or HEC Carboxymethyl or Hydroxyethyl or ester modified cellulose.
or EMC
SS Agglom. Suds Suppressor agglomerate: 12% Silicone/silica, 18% stearyl alcohol,70% starch in granular form.
TEPAE Tetreaethylenepentaamine ethoxylate.
pH Measured as a 1% solution in distilled water at 20° C.

### Example A

Bleaching detergent compositions having the form of granular laundry detergents are exemplified by the following formulations.

| | A | B | C | D | E | F |
|---|---|---|---|---|---|---|
| LAS | 20 | 22 | 20 | 15 | 20 | 20 |
| QAS | 0.7 | 1 | 1 | 0.6 | 0.0 | 0.7 |
| C25E3S | 0.9 | 0.0 | 0.9 | 0.0 | 0.0 | 0.9 |
| C25E7 | 0.0 | 0.5 | 0.0 | 1 | 3 | 1 |
| STPP | 23 | 30 | 23 | 17 | 12 | 23 |
| Zeolite A | 0.0 | 0.0 | 0.0 | 0.0 | 10 | 0.0 |
| Silicate | 7 | 7 | 7 | 7 | 7 | 7 |
| Carbonate | 15 | 14 | 15 | 18 | 15 | 15 |
| AA Polymer | 1 | 0.0 | 1 | 1 | 1.5 | 1 |
| CMC | 1 | 1 | 1 | 1 | 1 | 1 |
| Protease 32.89 mg/g | 0.1 | 0.07 | 0.1 | 0.1 | 0.1 | 0.1 |
| Amylase 8.65 mg/g | 0.1 | 0.1 | 0.1 | 0.0 | 0.1 | 0.1 |
| Lipase 18 mg/g | 0.03 | 0.07 | 0.3 | 0.1 | 0.07 | 0.1 |
| Brightener-Tinopal AMS (Ciba) | 0.06 | 0.0 | 0.06 | 0.18 | 0.06 | 0.06 |
| Brightener-Tinopal CBS-X (Ciba | 0.1 | 0.06 | 0.1 | 0.0 | 0.1 | 0.1 |
| DTPA | 0.6 | 0.3 | 0.6 | 0.25 | 0.6 | 0.6 |
| MgSO₄ | 1 | 1 | 1 | 0.5 | 1 | 1 |
| PC3 | 0.0 | 5.2 | 0.1 | 0.0 | 0.0 | 0.0 |
| PB1 | 4.4 | 0.0 | 3.85 | 2.09 | 0.78 | 3.63 |
| NOBS | 1.9 | 0.0 | 1.66 | 1.77 | 0.33 | 0.75 |
| TAED | 0.58 | 1.2 | 0.51 | 0.0 | 0.015 | 0.28 |
| Sulphate/Moisture | Balance | Balance | Balance | Balance | Balance | Balance |
| | to 100% | to 100% | to 100% | to 100% | to 100% | to 100% |

Any of the compositions in Example A is used to launder fabrics at a concentration of 600-10000 ppm in water, with typical median conditions of 2500 ppm, 25° C., and a 25:1 water:cloth ratio. The typical pH is about 10 but can be can be adjusted by altering the proportion of acid to Na— salt form of alkylbenzenesulfonate.

### Example B

Bleaching detergent compositions having the form of granular laundry detergents are exemplified by the following formulations.

| | A | B | C | D |
|---|---|---|---|---|
| LAS | 8 | 7.1 | 7 | 6.5 |
| C25E3S | 0 | 4.8 | 0 | 5.2 |
| C68S | 1 | 0 | 1 | 0 |
| C25E7 | 2.2 | 0 | 3.2 | 0 |
| QAS | 0.75 | 0.94 | 0.98 | 0.98 |
| (Na-)SKS-6 | 4.1 | 0 | 4.8 | 0 |
| Zeolite A | 20 | 0 | 17 | 0 |
| Citric | 3 | 5 | 3 | 4 |
| Carbonate | 15 | 20 | 14 | 20 |
| Silicate | 0.08 | 0 | 0.11 | 0 |
| Soil release agent | 0.75 | 0.72 | 0.71 | 0.72 |
| MA/AA | 1.1 | 3.7 | 1.0 | 3.7 |
| CMC | 0.15 | 1.4 | 0.2 | 1.4 |
| Protease | 0.37 | 0.4 | 0.4 | 0.4 |
| (56.00 mg active/g) | | | | |
| Termamyl | 0.3 | 0.3 | 0.3 | 0.3 |
| (21.55 mg active/g) | | | | |
| Lipase | 0.05 | 0.15 | 0.1 | 0.5 |
| (18.00 mg active/g) | | | | |
| Amylase | 0.1 | 0.14 | 0.14 | 0.3 |
| (8.65 mg active/g) | | | | |
| TAED | 3.6 | 4.0 | 3.6 | 4.0 |
| PC3 | 13 | 13.2 | 13 | 13.2 |
| EDDS | 0.2 | 0.2 | 0.2 | 0.2 |
| HEDP | 0.2 | 0.2 | 0.2 | 0.2 |
| MgSO₄ | 0.42 | 0.42 | 0.42 | 0.42 |
| Perfume | 0.5 | 0.6 | 0.5 | 0.6 |
| SS Agglom. | 0.05 | 0.1 | 0.05 | 0.1 |
| Soap | 0.45 | 0.45 | 0.45 | 0.45 |
| Sulphate | 22 | 33 | 24 | 30 |
| Water & Miscellaneous | Balance to | Balance to | Balance to | Balance to |
| | 100% | 100% | 100% | 100% |

Any of the above compositions in Example B is used to launder fabrics at a concentration of 10,000 ppm in water, 20-90° C., and a 5:1 water:cloth ratio. The typical pH is about 10 but can be can be adjusted by altering the proportion of acid to Na-salt form of alkylben zenesulfon ate.

Example C

| | A | B | C | D | E | F |
|---|---|---|---|---|---|---|
| | (wt %) | (wt %) | (wt %) | (wt %) | (wt %) | (wt %) |
| C25E1.8S | 11 | 10 | 4 | 6.32 | 6.0 | 8.2 |
| LAS | 4 | 5.1 | 8 | 3.3 | 4.0 | 3.0 |
| Sodium formate | 1.6 | 0.09 | 1.2 | 0.04 | 1.6 | 1.2 |
| Sodium hydroxide | 2.3 | 3.8 | 1.7 | 1.9 | 2.3 | 1.7 |
| Monoethanolamine | 1.4 | 1.490 | 1.0 | 0.7 | 1.35 | 1.0 |
| Diethylene glycol | 5.5 | 0.0 | 4.1 | 0.0 | 5.500 | 4.1 |
| C23E9 | 0.4 | 0.6 | 0.3 | 0.3 | 2 | 0.3 |
| DTPA | 0.15 | 0.15 | 0.11 | 0.07 | 0.15 | 0.11 |
| Citric Acid | 2.5 | 3.96 | 1.88 | 1.98 | 2.5 | 1.88 |
| C₁₂₋₁₄ dimethyl | 0.3 | 0.73 | 0.23 | 0.37 | 0.3 | 0.225 |
| Amine Oxide | | | | | | |
| C₁₂₋₁₈ Fatty Acid | 0.8 | 1.9 | 0.6 | 0.99 | 0.8 | 0.6 |
| Borax | 1.43 | 1.5 | 1.1 | 0.75 | 1.43 | 1.07 |
| Ethanol | 1.54 | 1.77 | 1.15 | 0.89 | 1.54 | 1.15 |
| TEPAE¹ | 0.3 | 0.33 | 0.23 | 0.17 | 0.0 | 0.0 |
| ethoxylated hexamethylene diamine² | 0.8 | 0.81 | 0.6 | 0.4 | 0.0 | 0.0 |
| 1,2-Propanediol | 0.0 | 6.6 | 0.0 | 3.3 | 0.0 | 0.0 |
| Protease* | 36.4 | 36.4 | 27.3 | 18.2 | 36.4 | 27.3 |
| Mannanase* | 1.1 | 1.1 | 0.8 | 0.6 | 1.1 | 0.8 |
| Amylase* | 7.3 | 7.3 | 5.5 | 3.7 | 7.3 | 5.5 |
| Lipase* | 10 | 3.2 | 0.5 | 3.2 | 2.4 | 3.2 |
| Water, perfume, dyes & | Balance | Balance | Balance | Balance | Balance | Balance |
| other components | | | | | | |

*Numbers quoted in mg enzyme/100 g
¹as described in US 4,597,898.
²available under the tradename LUTENSIT ® from BASF and such as those described in WO 01/05874

All documents cited in the Detailed Description of the Invention are in relevant part incorporated herein by reference: the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are

What is claimed is:

1. A composition comprising a detergent ingredient and a variant of a parent lipase, said variant, when compared to said parent, comprising a total of at least three substitutions, said substitutions being selected from one or more of the following groups of substitutions:
a) at least two substitutions in Region I,
b) at least one substitution in Region II,
c) at least one substitution in Region III, and/or
d) at least one substitution in Region IV.

2. A detergent composition according to claim 1, wherein said substitutions in Region I comprise substitutions in the positions corresponding to the positions 231 and 233.

3. A detergent composition according to claim 2 wherein said substitutions at positions 231 and 233 are substituted with an R.

4. A detergent composition according to claim 2, wherein said variant comprises a substitution in the position corresponding to position 4 of SEQ ID NO:2.

5. A detergent composition according to claim 4 wherein said substitution in the position corresponding to position 4 of SEQ ID NO:2 is V.

6. A detergent composition according to claim 2, wherein said variant comprises a substitution in the corresponding to position 227 of SEQ ID NO:2.

7. A detergent composition according to claim 6 wherein said substitution in the position corresponding to position 227 of SEQ ID NO:2 is G.

8. A detergent composition according to claim 1, wherein said at least one substitution in Region II comprises a substitution selected from the group consisting of substitutions in positions corresponding to the positions 202, 211, 255 and 256.

9. A detergent composition according to claim 8, wherein said at least one substitution in Region II comprises a substitution selected from the group consisting of X202G, X211L, X255Y/V and X256K.

10. A detergent composition according to claim 1, wherein said at least one substitution in Region II comprises a substitution in the position corresponding to the position 210.

11. A detergent composition according to claim 10, wherein said substitution corresponding to position 210 comprises X210K.

12. A detergent composition according to claim 1, wherein said at least one substitution in Region III comprises a substitution selected from the group consisting of substitutions in positions corresponding to the positions 83, 86 and 90.

13. A detergent composition according to claim 11, wherein said at least one substitution in Region III comprises a substitution selected from the group consisting of X83T, X86V and X90A/R.

14. A detergent composition according to claim 1, wherein said at least one substitution in Region III comprises a substitution in the position corresponding to the position 83.

15. A detergent composition according to claim 14, wherein said substitution corresponding to position 83 comprises X83T.

16. A detergent composition according to claim 1, wherein said at least one substitution in Region IV comprises a substitution selected from the group consisting of substitutions in positions corresponding to the positions 27, 58 and 60.

17. A detergent composition according to claim 16, wherein said at least one substitution in Region IV comprises a substitution selected from the group consisting of X27R, X58N/A/G/P/T and X60S/V/G/N/R/K/A/L.

18. A detergent composition according to claim 1, comprising at least two substitutions in Region IV corresponding to the positions 27, 58 and 60.

19. A detergent composition according to claim 1, comprising at least two substitutions in Region IV selected from the group consisting of X27R, X58N/A/G/P/T and X60S/V//G/N/RK/A/L.

20. A detergent composition according to claim 1, wherein said variant comprises at least one substitution outside the defined Regions I to IV.

21. A detergent composition according to claim 20, wherein said at least one substitution outside the defined Regions I to IV is selected from the group consisting of substitutions in positions corresponding to position 81, 147, 150 and 249.

22. A detergent composition according to claim 20, wherein said at least one substitution outside the defined Regions I to IV is selected from the group consisting of X81Q/E, X147M/Y, X150G and X249R/I/L.

23. A detergent composition according to claim 2, wherein said parent lipase is at least 90% identical to SEQ ID NO:2.

24. A detergent composition according to claim 1 wherein the parent lipase is identical to SEQ ID NO: 2 and said variant comprises one of the following groups of substitutions:
a) T231R+N231R+1255Y
b) 1202G+T231R+N233R
c) 186V+L227G+T231R+N233R+P256K
d) Q4V+S58N+V60S+T231R+N233R
e) S58N+V60S+I90R+T231R+N233R
f) 190A+T231R+N231R+1255V
g) S58N+V60S+186V+A150G+L227G+T231R+N233R+P256K
h) S58N+V60S+L147M+F211L+T231R+N231R
i) Q4V+S58A+V60S+S83T+186V+A150G+E210K+L227G+T231R+N233R+P256K
j) S58N+V60S+186V+A1SOG+L227G+T231R+N233R+P256K.

25. A detergent composition according to claim 1 wherein the parent lipase is identical to SEQ ID NO: 2 and said variant comprises one of the following groups of substitutions:
a) Q4V+S58A+V60S+S83T+I86V+A150G+E210K+L227G+T231R+N233R+P256K
b) S58N+V60S+I86V+A150G+L227G+T231R+N233R+P256K.

26. A detergent composition according to claim 1 wherein the lipase variant is characterized in that the Benefit Risk (BR), when measured as given in the specification, is larger than 1.

27. A detergent composition according to claim 1 further comprising 0.1 to 40% anionic surfactant, preferably from 0.1 to 12%.

28. A detergent composition according to claim 27 wherein the anionic surfactant is a alkoxylated alkyl sulphate.

29. A detergent composition according to claim 1 further comprising 5 to 30% aluminosilicate and/or phosphate builder.

30. A detergent composition according to claim 1 further comprising a source of peroxide and a bleach activator, preferably.

31. A detergent according to claim 1 wherein said detergent is a liquid detergent composition or a solid detergent composition.

32. A detergent according to claim 31 wherein said detergent is a granular detergent.

33. A detergent according to claim 1 wherein said detergent is a unit dose composition that is a solid tablet or a liquid encapsulated in a soluble film.

34. A washing process comprising laundering textile articles in an aqueous solution comprising the detergent composition according to claim 1.

35. A washing process according to claim 34 to of removing soils and stains from a surface comprising the steps of:
a) optionally pretreating the soils and stains with the compositions of claim 1 to form an optionally pretreated surface;
b) adding an effective amount of the compositions of claim 1 to water to form from an aqueous washing solution comprising about 500 to about 10000 ppm of the composition;
c) contacting the aqueous washing solution with the optionally pretreated surface, and d) optionally providing agitation to the aqueous washing solution and the optionally pretreated surface.

36. A washing process according to claim 34 in which the aqueous solution is at a temperature below 30° C.
